(19) United States
(12) Patent Application Publication
(10) Pub. No.: US 2009/0204190 A1
(43) Pub. Date: Aug. 13, 2009

(54) Stanan Kozy Pak Ice Holder

(76) Inventor: Nancy Ellen Sestito, Morgan Hill, CA (US)

Correspondence Address: Nancy Sestito, 19067 Savannah Court, Morgan Hill, CA, 95037, US

(21) Appl. No.: 12/069,208

(22) Filed: Feb. 9, 2008

(51) Int. Cl. A61F 7/00

(52) U.S. Cl. 607/108; 607/112; 607/114

(57) ABSTRACT

The stanan Kozy Pak Holder is a new way to apply ice to areas of the body to prevent bruising and selling due to surgery or injury. The major benefits are the stanan Kozy Pak Holder is that it is more comfortable to use and maintains the coldness required to reduce bruising and swelling longer then any ice pack on the market.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION:

The stanan Kozy Pac Holder is for medical use and was invented to significantly reduce swelling and bruising due to surgery or injury by enabling ice cubes to be placed in standard quart size plastic baggies. The ice filled plastic baggies are then placed inside one or more of the three available pockets depending on how much body area requires ice packs.

### BRIEF SUMMARY OF THE INVENTION

The stanan Kozy Pac Holder enables consumers to use ice cubes that are placed in standard quart sized plastic baggies and the plastic baggies are then placed inside the pockets of the stanan Kozy Pak Holder. The filled stanan Kozy Pak Holder is then placed on the body where surgery was performed or an injury occurred to significantly reduce bruising and swelling.

### BRIEF DESCRIPTION OF THE SEVERAL VIEWS OF THE DRAWINGS

FIG. 1: View of piece of terry cloth that is the starting point showing the terry cloth as one large piece measuring 22.5 inches high×27 inches wide.

FIG. 2: Back view of the single piece of terry cloth with small hook Velcro strips (3″×2″) sowed on.

FIG. 3: Back view of pockets with hook Velcro and 6 inch strip of soft Velcro tab sown into left edge seam.

FIG. 4: Front view of the stanan Kozy Pak Holder with pockets sowed.

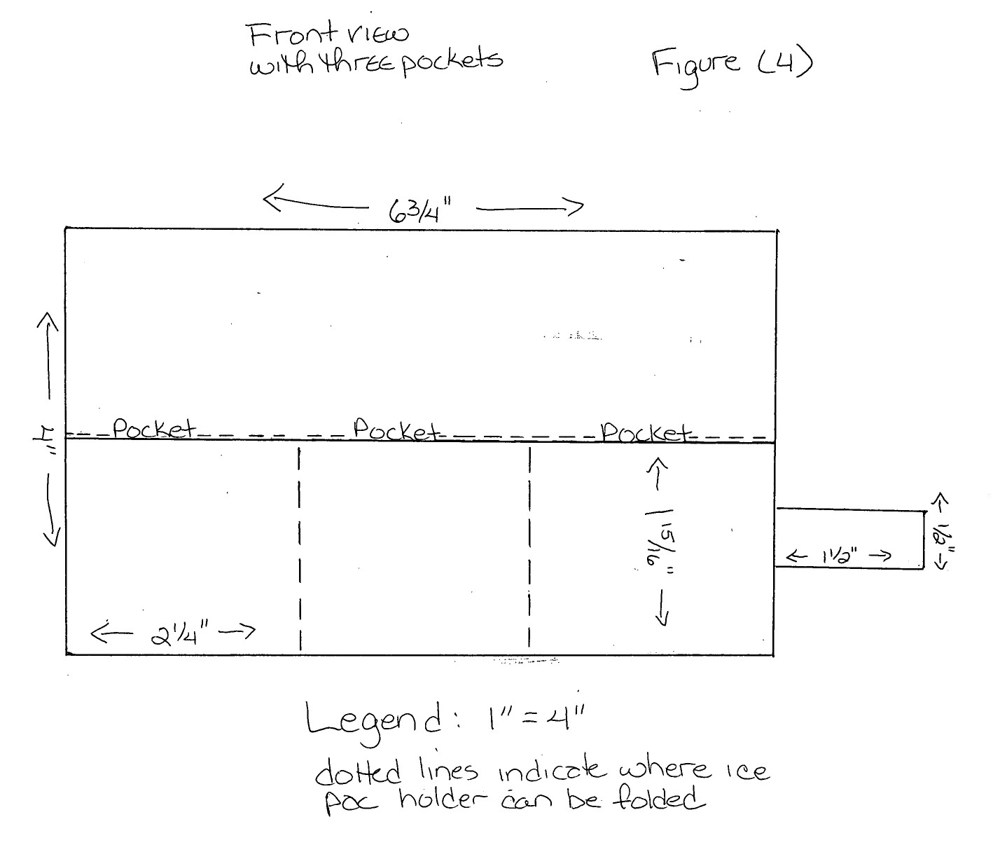

FIG. 5: Front view with flap folder over pockets.

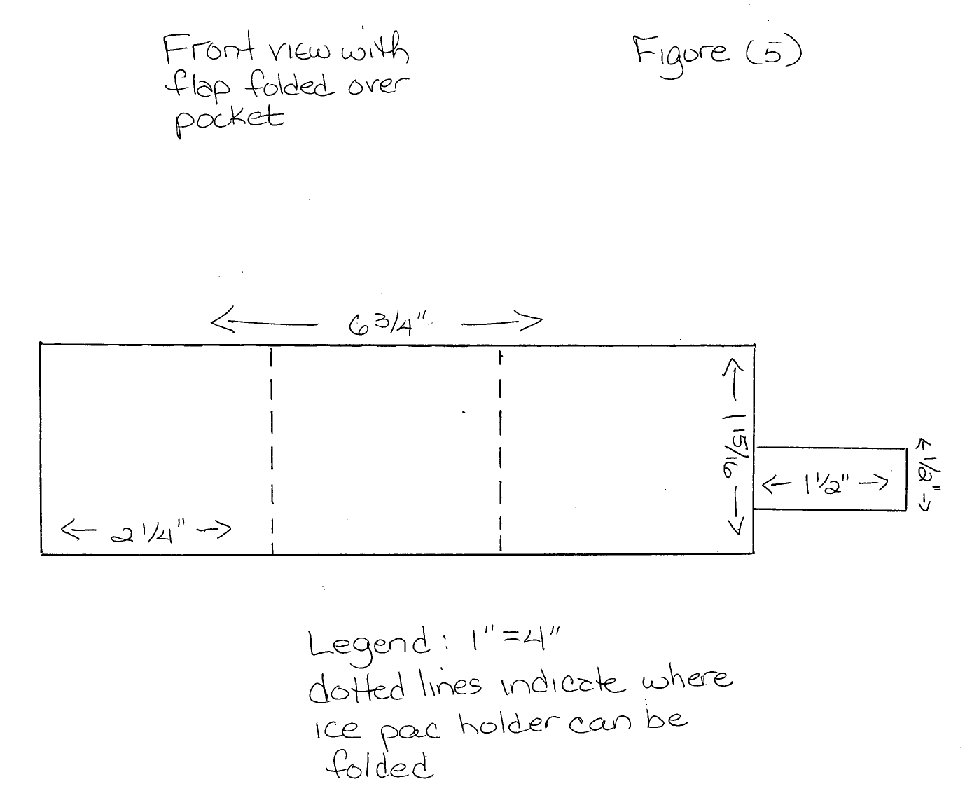

FIG. 6: Back view with flap folder over front pockets.

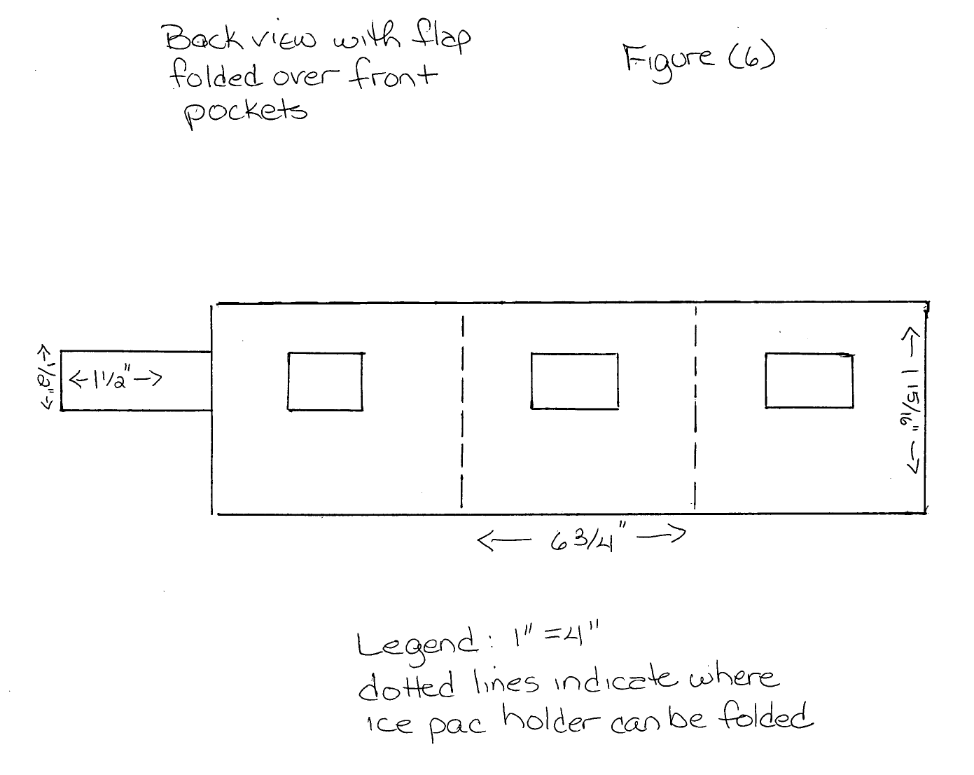

### DETAILED DESCRIPTION OF THE INVENTION

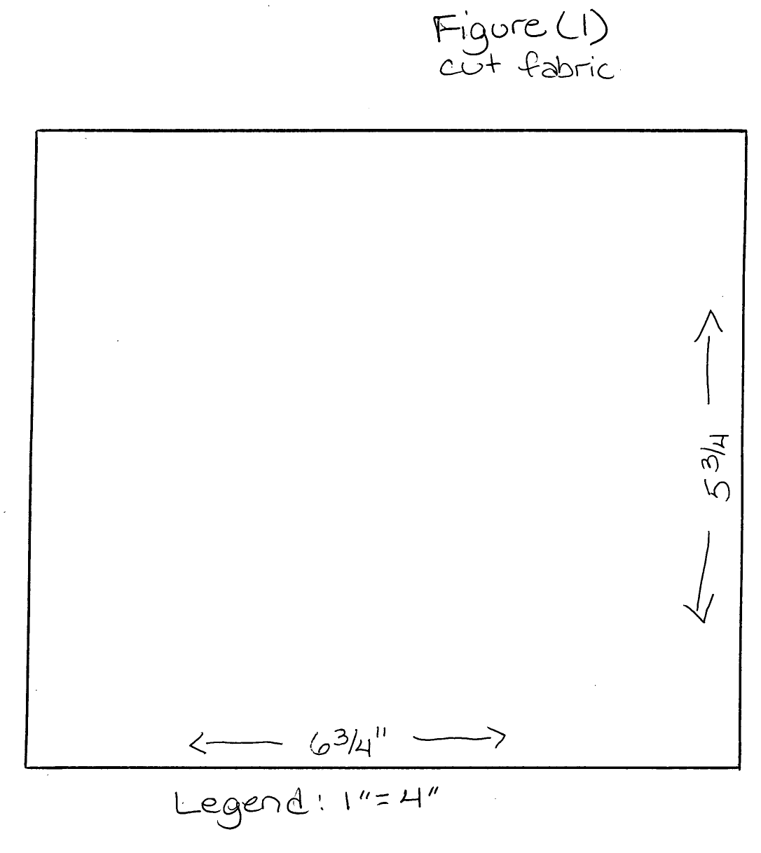

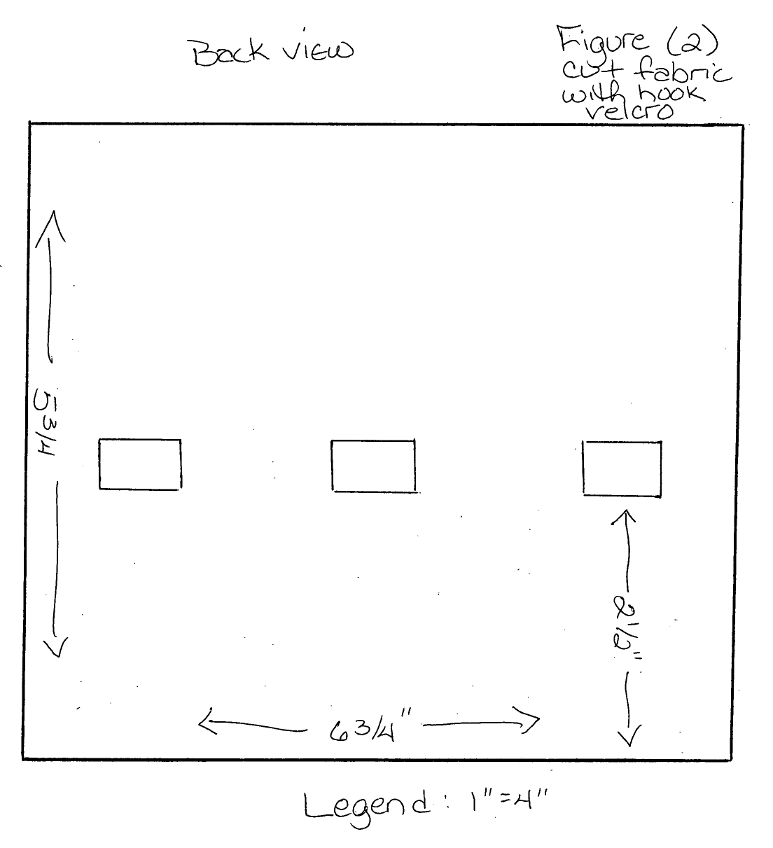

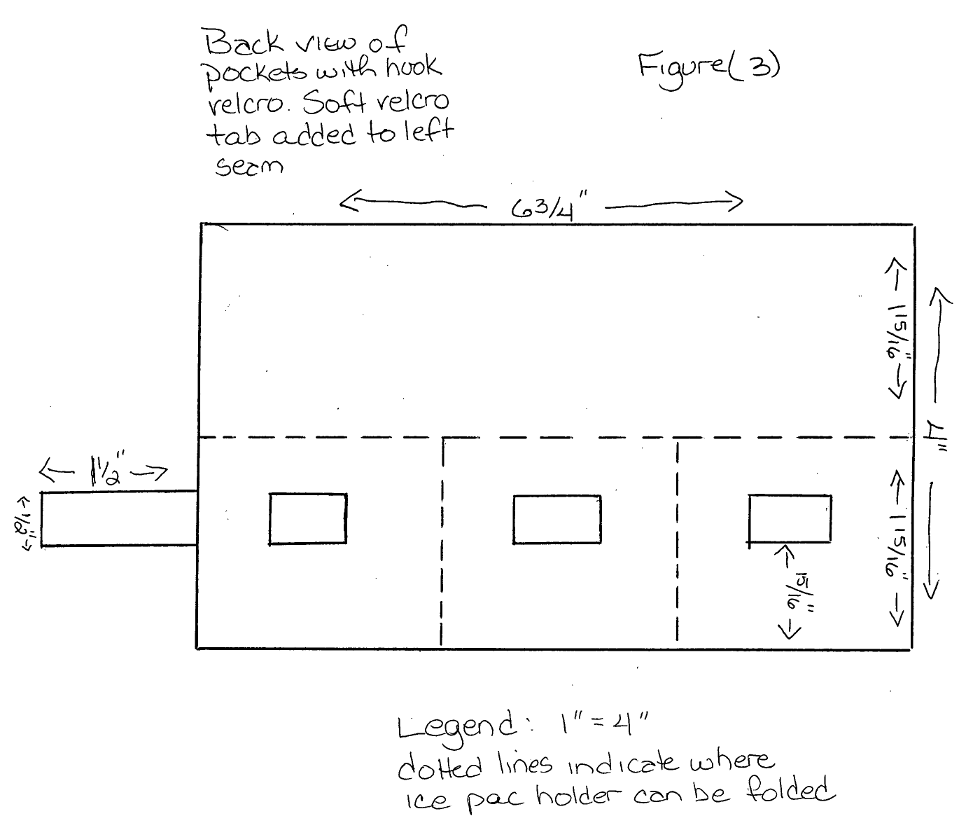

The stanan Kozy Pak Ice Holder is made by starting with a 22.5 inches high by 27 inches wide piece of terry cloth (FIG. 1). The first thing is the terry cloth has the three unfinished sides of its perimeter sown with an over lock stitch to prevent fraying, one side has the salvage edge. Then 10 inches from the bottom 3 pieces of Velcro are sown to the back of the terry cloth with each piece of Velcro 3 inches long by 2 inches wide (FIG. 2). The first piece of Velcro is sown from the edge of the right side of the terry cloth extending to 3 inches from the right side edge. The second piece of Velcro is sown 12 inches from right edge. The 3^{rd} piece of Velcro is sown 3 inches from the edge of the left edge. The left edge of the terry cloth (when folded over to form pockets) will have a 6 inch piece of Velcro sown into the seam and this piece of Velcro will be able to attach to any of the 3, three inch pieces of Velcro, depending on what size is needed. This is what holds the stanan Kozy Pak Ice Holder in place. The next step is to take the bottom of the terry cloth and fold it up to form a 7½ inch pocket. Then the right and left edges of the pockets are sown. Then 9 inches from the right edge and 9 inches from the left edge is sown to complete the 3 pockets which will house the standard quart size plastic baggies filled with ice cubes. (FIG. 3) The purpose for this terry cloth ice pack holder with 3 removable plastic bags is so enough ice can be placed in the pockets to keep the temperature low enough and long enough to have a positive effect when placed on the swelling and bruising for a long period of time, up to twelve (12) hours without changing the ice when all three pockets are filled with ice. This will allow for a longer uninterrupted periods of rest. The ice pack holder is made of soft terry cloth, it is comfortable and aids in absorbing the condensation so you stay dry (the stanan Kozy Pak can be put into the clothes dryer if it gets wet). It also, prevents ice burns to the skin. The Velcro closures are strong enough to keep it in place if you have to leave the bed. It is no longer painful to keep an area of the skin iced for a long period of time.

It is designed for your face after face lift surgery: one pocket keeps the right side of the face cold, one pocket keeps the under side of the chin cold and one pocket keeps the left side of the face cold. It is also wide enough that when placed on the head, the cold radiates to the cheeks. It is also designed and can be used with 1, 2 or 3 packs of ice for applying to different parts of your body after surgery or an injury. It can used on the head, face, legs, stomach, backside, back, shoulder and feet.

The pockets are design to accommodate standard quart size plastic baggies, either food storage or freezer type plastic baggies which are available at any food or convenience store.

1. The stanan Kozy Pak Holder allows users to apply ice on areas of the body, due to surgery or injury, which reduces bruising and swelling. When all 3 pockets are filled with ice cubes placed inside quart sized plastic baggies it will maintain the coldness for up to twelve (12) hours.

2. The terry cloth keeps the skin comfortable and dry and allows a user to keep it on for up to twelve (12) hours. This allows the user to keep the stanan Kozy Pak on and sleep through the night.
